(19) United States
(12) Patent Application Publication
(10) Pub. No.: US 2024/0036049 A1
(43) Pub. Date: Feb. 1, 2024

(54) BIOMARKER, FOR DIAGNOSING PANCREATIC CANCER, COMPRISING ASPROSIN, AND USE THEREOF

(71) Applicants: SEOUL NATIONAL UNIVERSITY R&DB FOUNDATION, Seoul (KR); INHA-INDUSTRY PARTNERSHIP INSTITUTE, Incheon (KR)

(72) Inventors: Sunghyouk PARK, Seoul (KR); Sunmi KANG, Seoul (KR); Hoonsik NAM, Seoul (KR); Soon Sun HONG, Seoul (KR); Kyung Hee JUNG, Bucheon-si, Gyeonggi-do (KR)

(73) Assignees: SEOUL NATIONAL UNIVERSITY R&DB FOUNDATION, Seoul (KR); INHA-INDUSTRY PARTNERSHIP INSTITUTE, Incheon (KR)

(21) Appl. No.: 18/265,677

(22) PCT Filed: Apr. 8, 2021
(86) PCT No.: PCT/KR2021/004403

§ 371 Date: Jun. 7, 2023

(30) Foreign Application Priority Data
Dec. 10, 2020 (KR) 10-2020-0171935

(51) Int. Cl. G01N 33/574

(52) U.S. Cl. CPC G01N 33/57438; G01N 2333/575

(57) ABSTRACT

A biomarker, for diagnosing pancreatic cancer, including asprosin, and the use thereof, has been confirmed that: the survival prognosis of a pancreatic cancer patient is worse in the case of a higher expression of FBN-1 gene encoding fibrillin-1 which is the precursor of asprosin in a clinical information database; an asprosin level at a cell level is related to an occurrence of pancreatic cancer; and the asprosin level in the blood of a pancreatic cancer patient or an early stage pancreatic cancer patient is higher than that of a healthy person.

### TECHNICAL FIELD

The present disclosure relates to a biomarker for diagnosing pancreatic cancer comprising asprosin and a use thereof.

### BACKGROUND ART

Pancreatic cancer is one of cancers with a low survival rate within 5 years due to difficulty in early detection and treatment. According to a report of the Ministry of Health and Welfare in 2019, the number of pancreatic cancer patients in 2017 was 7,032 per 100,000 people with an increase of 4.6% compared to the previous year, and the number of cases by cancer type rose to 8^{th} place. The incidence of pancreatic cancer has also been on the rise since 1999. Thyroid cancer and breast cancer show high survival rates of 100.1% and 93.2%, respectively, and the survival rate of many cancers is increasing, while pancreatic cancer has a relatively low survival rate of 12.2%.

To date, there is no diagnostic method specific to pancreatic cancer, making diagnosis difficult at an early stage. There are many cases that examination at the early stage of cancer is missed out since detection and classification of symptoms are difficult even with the aid of abdominal ultrasound, abdominal CT, and MRI scanning. A level of carbohydrate antigen 19-9 (CA 19-9), a blood tumor marker, may increase in pancreatic cancer patients, but it also increases in patients with pancreatitis or biliary obstruction to have limitation in the diagnosis of pancreatic cancer due to inability to distinguish from other cancers such as colorectal cancer and breast cancer, such that it is not recommended in the diagnosis.

As such, pancreatic cancer is not only difficult to diagnose early but also known to have poor prognosis with a low survival rate of 12.2% due to problems such as resistance to anticancer drugs, metastasis to other organs, and limited range of treatment by surgical operation of only about 10 to 20%, even if it is identified as pancreatic cancer.

Therefore, there is an urgent need for research on methods that enable effective diagnosis by discovering pancreatic cancer-specific biomarkers capable of accurately diagnosing pancreatic cancer at an early stage.

### PRIOR ART DOCUMENT

### Patent Document

Korean Patent Application Publication No. 10-2015-0001287 (published on Jan. 6, 2015)

### DISCLOSURE OF THE INVENTION

### Technical Goals

The present disclosure relates to a biomarker for diagnosing pancreatic cancer comprising asprosin and a use thereof, and an object of the present disclosure is to provide asprosin as a biomarker for pancreatic cancer diagnosis and post-treatment prognosis assessment, by identifying that survival prognosis of pancreatic cancer patients becomes worse with the higher expression of an FBN-1 gene that encodes fibrillin-1, a precursor of asprosin, in the clinical information database, determining that a level of asprosin at a cellular level is associated with the development of pancreatic cancer, and finding that the level of asprosin in the blood of patients with pancreatic cancer and early-stage pancreatic cancer is higher than that of healthy individuals.

### Technical Solutions

The present disclosure provides a biomarker composition for diagnosing pancreatic cancer, comprising asprosin as an active ingredient.

In addition, the present disclosure provides a composition for diagnosing pancreatic cancer, comprising a preparation for measuring a level of asprosin.

In addition, the present disclosure provides a kit for diagnosing pancreatic cancer, comprising the composition for diagnosing pancreatic cancer.

In addition, the present disclosure provides a method of providing information for diagnosis and prognosis assessment of pancreatic cancer, comprising measuring a level of asprosin in a biological sample derived from a subject; determining, if the level of asprosin is higher than that of a healthy control group, that pancreatic cancer has developed or is likely to develop; determining, if the level of asprosin during chemotherapy in the subject or after the chemotherapy appears to be lower than that before the chemotherapy, that there is improvement in the pancreatic cancer of the subject; and determining that a survival rate is higher as the level of asprosin is lower during the chemotherapy or after the chemotherapy.

### Advantageous Effects

According to the present disclosure, it is possible to provide asprosin as a biomarker for diagnosis and prognosis assessment of pancreatic cancer, by identifying that the survival prognosis of pancreatic cancer patients becomes worse with the higher expression of an FBN-1 gene that encodes fibrillin-1, a precursor of asprosin, in the clinical information database, determining that a level of asprosin at a cellular level is associated with the development of pancreatic cancer, and finding that the level of asprosin in the blood of patients with pancreatic cancer and early-stage pancreatic cancer is higher than that of healthy individuals.

### BRIEF DESCRIPTION OF DRAWINGS

FIG. 1 shows a schematic diagram in which 140 amino acids in C-terminus of profibrillin-1 encoded by an FBN-1 gene are cleaved to form asprosin.

FIG. 2 shows a result of analyzing mRNA expression of an FBN1 gene in normal and cancer cell lines through the Gene Expression Profiling Interactive Analysis (GEPIA) web database.

FIG. 3 shows results of analyzing mRNA expression of an FBN1 gene in pancreatic cancer sets from the Cancer Genome Atlas (TCGA) clinical and genomic information database.

FIG. 4 shows results of measuring a level of asprosin in a pancreatic cancer cell line.

FIG. 5 shows a result of measuring a level of asprosin in the blood of normal and pancreatic cancer patients.

FIG. 6 is a result of analyzing the level of asprosin in the FIG. 5 by Receiver Operating Characteristic (ROC).

FIG. 7 shows a result of measuring a level of asprosin in the blood of normal and early-stage pancreatic cancer (AJCC stages 1 and 2) patients.

FIG. 8 shows a result of analyzing the level of asprosin in the FIG. 7 by Receiver Operating Characteristic (ROC).

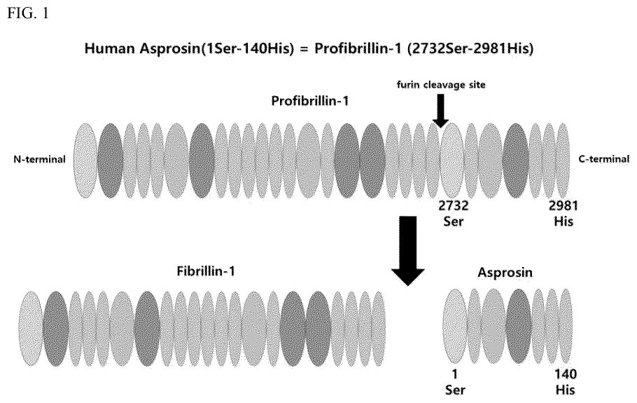

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

The numerical range includes the numerical value defined in the above range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Hereinafter, the present disclosure will be described in more detail.

The present disclosure provides a biomarker composition for diagnosis and prognosis assessment of pancreatic cancer, including asprosin as an active ingredient.

The diagnosis includes determining of susceptibility to a particular disease or disorder of an individual, determining whether an individual currently has a particular disease or disorder, determining of prognosis of an individual having a particular disease or disorder (e.g., identification of a pre-metastatic or metastatic state of cancer, determination of the stage of cancer, or determination of reactivity to treatment of a cancer), or therametrics (e.g., monitoring of a state of a subject to provide information on therapeutic efficacy). For the purposes of the present disclosure, the diagnosis is to identify whether pancreatitis has developed or is likely to develop.

The biomarker is a substance to diagnose pancreatic cancer and typically includes organic biomolecules such as polypeptides, nucleic acids (e.g., mRNA, etc.), lipids, glycolipids, glycoproteins, and sugars (monosaccharides, disaccharides, oligosaccharides, etc.), but in the present disclosure, it is an asprosin hormone protein.

The asprosin is a protein including 140 amino acids formed by cleavage of the C-terminal region in profibrillin-1, specifically amino acids represented by SEQ ID NO: 1.

The pancreatic cancer is an early-stage pancreatic cancer diagnosed as Stage 1 or Stage 2 by American Joint Committee on Cancer (AJCC) classification.

In addition, the present disclosure provides a composition for diagnosing pancreatic cancer, including a preparation for measuring a level of asprosin.

The preparation for measuring the level of asprosin is an antibody that specifically binds to the asprosin, and in particular, the antibody specifically binding to the asprosin is commercially available, and specifically, an antibody of Asprosin (ASPROSIN), ELISA Kit (MBS167434) from Mybiosource (CA, USA) may be used.

The antibody refers to a specific protein molecule indicated for an antigenic site, and includes all polyclonal antibodies, monoclonal antibodies, and recombinant antibodies.

In addition, the present disclosure provides a kit for diagnosing pancreatic cancer, including the composition for diagnosing pancreatic cancer.

The kit may further include one or more other component compositions, solutions, or devices suitable for analytical methods, preferably in the form of a protein chip kit.

In addition, the present disclosure provides a method of providing information for diagnosis and prognosis assessment of pancreatic cancer, including measuring a level of asprosin in a biological sample derived from a subject; and determining, if the level of the asprosin is higher than that of a healthy control group, that pancreatic cancer has developed or is likely to develop.

The level of the asprosin may be measured by one or more methods selected from the group consisting of silver Western blot, enzyme linked immunosorbent assay (ELISA), radioimmunoassay (RIA), radioimmunodiffusion, Ouchterlony immunodiffusion, rocket immunoelectrophoresis, immunohistochemistry, immunoprecipitation assay, complement fixation assay, fluorescence activated cell sorter (FACS) and protein chip.

The biological sample may be blood, plasma, or urine. The method of providing information further includes determining that survival prognosis of a subject is worse as the level of the asprosin is higher.

The method of providing information includes determining, if the level of asprosin during the chemotherapy or after the chemotherapy appears to be lower than that before the chemotherapy in the subject, that there is improvement in the pancreatic cancer of the subject.

The method of providing information includes determining that a survival rate is higher as the level of asprosin is lower during chemotherapy in the subject or after the chemotherapy.

### MODES FOR CARRYING OUT THE INVENTION

Hereinafter, in order to help the understanding of the present disclosure, experimental examples and example embodiments will be described in detail. However, the following experimental examples and example embodiments are merely illustrative of the present disclosure, and the scope of the present disclosure is not limited to the following experimental examples and example embodiments. Experimental examples and example embodiments of the present disclosure are provided to more completely explain the present disclosure to those skilled in the art.

### <Experimental Example> Experimental Materials and Methods

The following Experimental Examples are intended to provide Experimental Examples commonly applied to each Example Embodiment according to the present disclosure.

1. Analysis of Genetic Data

Gene Expression Profiling Interactive Analysis (GEPIA) is a web server database enabling comparison and analysis of gene expression profiles of samples of cancer tissues and normal tissues based on The Cancer Genome Atlas (TCGA) and Genotype-Tissue Expression (GTEx). By searching for FBN1 (Ensembl ID: ENSG00000166147.13) on the GEPIA web server, the FNB1 gene expression profile was identified in whole cancer and normal tissues.

In addition, the FBN1 gene expression in the pancreatic cancer project file (TCGA-PAAD) of The Cancer Genome Atlas (TCGA) and clinical information of patients were analyzed. Based on overall survival rate (OS), disease-specific survival (DSS), progression-free interval (PFI), and disease-free interval (DFI) of the entire patients, groups were divided into a high group (FBN1 high) and a low group (FBN1 low) for FBN1 expression respectively to conduct survival analysis by a Kaplan-Meier plot.

2. Cell and Culture Conditions

Pancreatic cancer cell lines (BxPC3, MIA PaCa2, PANC1, HPAC, Aspc1) and normal pancreatic cell lines (HPNE) were purchased from the American Type Culture Collection (ATCC, Manassas, VA) and cultured in DMEM or RPMI media with 10% FBS and 1% penicillin/streptomycin (P/S) added. All cell lines were cultured in a humidity-controlled CO₂ incubator in the presence of 5% CO₂/95% air at 37° C. Cell culture media, FBS, penicillin-streptomycin and other supplements were purchased from GIBCO (Waltham, MA). Cells were seeded at 5×10⁴, and the amount of asprosin in the media after 48 h was identified using ELISA KIT. For asprosin staining in the cells, cells were seeded at 5×10⁴, fixed with ethanol, and washed with PBS. After immersing in 0.2% (v/v) Triton X-100 for 5 minutes at room temperature to make the cells permeable, asprosin (adipoGen, AG-20b-0073,1:50) primary antibodies were diluted in 3% BSA at 1:50, followed by incubation overnight at 4° C. After washing with PBS, the sample was incubated (1:100) at room temperature for 1 hour with secondary antibodies with biotin attached thereto and then washed 3 times with PBS. After reacting with ABC solution at room temperature for 1 hour, washing was performed, and a reaction was finally carried out in DAB solution to observe the staining of asprosin.

3. Blood Samples

Sera from 347 patients diagnosed with pancreatic cancer and a normal group of 200 individuals were obtained from biobanks of 11 hospitals in Korea. In addition, clinical epidemiological information such as gender, age, and stage was provided from pancreatic cancer patients, and information on gender and age from the normal group. All blood samples were bioresources obtained from biobanks in hospitals and used in experiments with the approval of the Institutional Bioethics Committee of Seoul National University R&DB Foundation. The provided sources, resources, clinical information, and the number of samples provided are shown in Table 1 below.

TABLE 1

| | | | | |
|---|---|---|---|---|
| | | | | Number |
| | Distributed | Distributed | Clinical | of |
| Source | disease | resources | Information | Samples |

Pancreatic cancer (n = 347)

| | | | | |
|---|---|---|---|---|
| Seoul National | Pancreatic | Serum | Gender, Age, | 40 |
| University Hospital- | cancer | | Stage | |
| Human Biobank | | | | |
| Kangwon National | Pancreatic | Serum | Gender, Age, | 20 |
| University Hospital- | cancer | | Stages (2) | |
| Human Biobank | | | | |
| Chungnam National | Pancreatic | Serum | Gender, Age | 20 |
| University Hospital- | cancer | | | |
| Human Biobank | | | | |
| Kyungpook National | Pancreatic | Serum | Gender, Age | 60 |
| University Hospital- | cancer | | | |
| Human Biobank | | | | |
| Keimyung University | Pancreatic | Serum | Gender, Age | 50 |
| Dongsan Hospital- | cancer | | | |
| Human Biobank | | | | |
| Gyeongsang National | Pancreatic | Serum | Gender, Age, | 40 |
| University Hospital- | cancer | | Stages (14) | |
| Human Biobank | | | | |
| Chungbuk National | Pancreatic | Serum | Gender, Age, | 49 |
| University Hospital- | cancer | | Stages (42) | |
| Human Biobank | | | | |
| Chonnam National | Pancreatic | Serum | Gender, Age, | 50 |
| University Hwasun | cancer | | Stages (45) | |
| Hospital-Human | | | | |
| Biobank | | | | |
| Inje University Busan | Pancreatic | Serum | Gender, Age, | 18 |
| Paik Hospital-Human | cancer | | Stages (15) | |
| Biobank | | | | |

Normal (n = 200)

| | | | | |
|---|---|---|---|---|
| Chungnam National | Normal group | Serum | Gender, Age | 20 |
| University Hospital- | | | | |
| Human Biobank | | | | |
| Ajou University | Normal group | Serum | Gender, Age | 40 |
| Hospital-Human | | | | |
| Biobank | | | | |
| Chungbuk National | Normal group | Serum | Gender, Age | 40 |
| University Hospital- | | | | |
| Human Biobank | | | | |
| Jeonbuk National | Normal group | Serum | Gender, Age | 50 |
| University Hospital- | | | | |
| Human Biobank | | | | |
| Kyungpook National | Normal group | Serum | Gender, Age | 50 |
| University Hospital- | | | | |
| Human Biobank | | | | |

4. ELISA Measurement

Human Asprosin ELISA kit was purchased from Mybiosource (CA, USA) to be used. Sera isolated from blood were added to a 96 well plate coated with asprosin primary antibodies (Mybiosource, for ELISA kit) by 50 ul each, and 50 ul of a standard protein was added to carry out a reaction at 37° C. for 2 hours. The samples and standard solutions were discarded, and 100 ul of detection Regent A was added, followed by a reaction at 37° C. for 1 hour. After discarding the reaction solution and washing with a washing buffer 3 times, 100 ul of detection Regent B was added to carry out a reaction at 37° C. for 1 hour. The reaction solution was discarded, washing was performed 5 times with a washing buffer, 90 ul of substrate was added, and a reaction was carried out for 10-30 minutes after blocking light. When color began to turn in blue, 50 ul of stop solution was added to stop the reaction, and the absorbance was measured at 450 nm using a spectrometer to measure the level of asprosin in the blood.

5. Statistical Analysis Methods

Using the concentration level of asprosin measured by the method 1 in the blood of the normal and the pancreatic cancer patients, the mean and standard deviation were obtained in the GraphPad Prism software, and unpaired t test with Welch's correction method was used to verify significance in the statistical difference between the normal and pancreatic cancer groups. Subsequently, Receiver Operating Characteristic (ROC) analysis was conducted to obtain the Area Under Curve (AUC) value, and sensitivity and specificity were calculated using the Euclidean method. The above analysis was also performed with asprosin values in the blood of the normal individuals and early-stage pancreatic cancer (AJCC Stage 1,2).

The prognosis stage groups were divided into stages 0˜IV according to primary tumor (T), regional lymph node metastasis (N), and distant metastasis (M) by the American Joint Committee on Cancer (AJCC) for pancreatic cancer. Stage I/IA/IB and IIA/IIB were classified as early-stage pancreatic cancer, and a comparative analysis on the entire pancreatic cancer groups and the early-stage pancreatic cancer groups was conducted compared to the normal.

### Example 1. Detection of Biomarkers for Diagnosing Pancreatic Cancer

In order to detect biomarkers capable of diagnosing pancreatic cancer, gene expression levels of pancreatic cancer cell lines and normal cell lines were analyzed in a genetic web database. The targets of analysis were adrenocortical carcinoma (ACC), bladder urothelial carcinoma (BLCA), breast invasive carcinoma (BRCA), cervical and endocervical cancer (CESC), cholangiocarcinoma (CHOL), colon adenocarcinoma (COAD), lymphoid neoplasm diffuse large B-cell lymphoma (DLBC), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), acute myeloid leukemia (LAML), brain lower grade glioma (LGG), liver hepatocellular carcinoma (LIHC), lung adenocarcinoma (LUAD), lung squamous cell carcinoma (LUSC), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), stomach adenocarcinoma (STAD), testicular germ cell tumors (TGCT), thyroid carcinoma (THCA), thymoma (THYM), uterine corpus endometrial carcinoma (UCEC), and uterine melanoma (UCS).

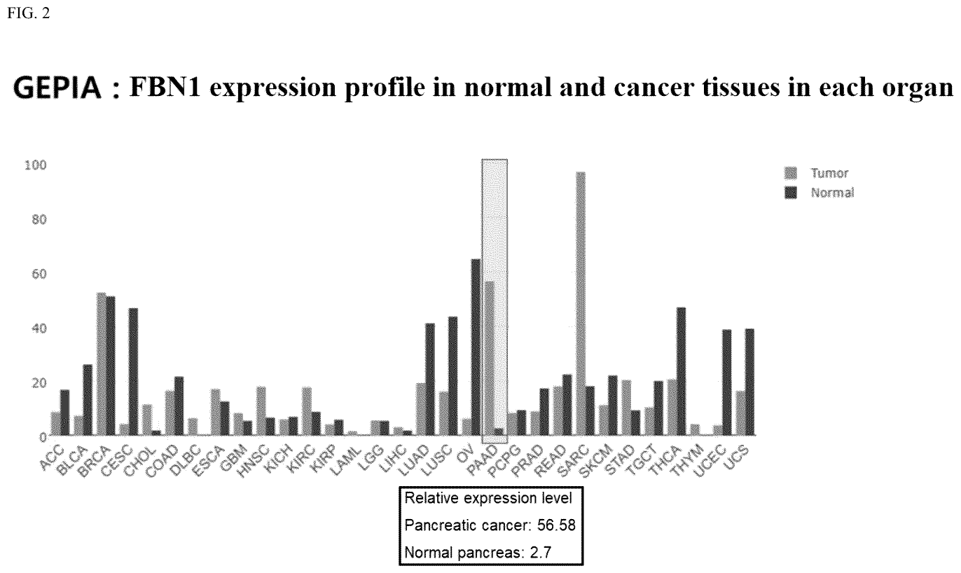

As shown in FIG. 2, as a result of analyzing mRNA expressed in various organ-derived normal cell lines and cancer cell lines in the Gene Expression Profiling Interactive Analysis (GEPIA) web database, expression of the FBN1 gene was higher in cancer cells than in normal cells only in pancreatic cancer and sarcoma. Specifically, for pancreatic cancer (PAAD), expression of FBN1 increased in cancer tissues compared to normal tissues, and the relative expression level was 21 times higher in the pancreatic cancer (56.58) than the normal pancreas (2.7).

In addition, for the FBN1 gene expression of the pancreatic cancer project file (TCGA-PAAD) of the Cancer Genome Atlas (TCGA) and clinical information on patients, analysis was conducted by dividing into the high group (FBN1 high) and the low group (FBN1 low) for FBN1 expression, respectively, based on the overall survival rate (OS), disease-specific survival (DSS), progression-free interval (PFI), and disease-free interval (DFI) of the entire patients.

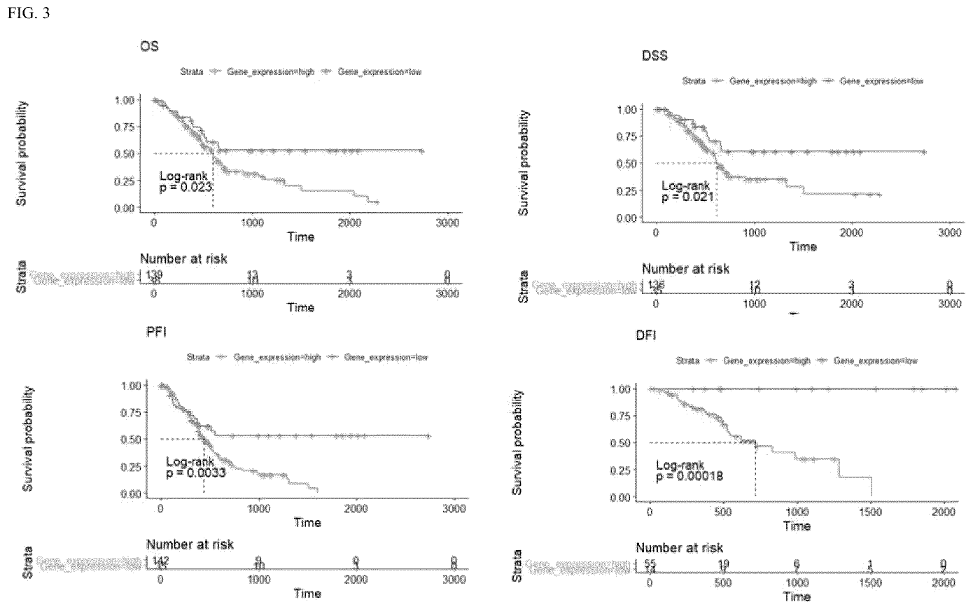

As shown in FIG. 3, as a result of analyzing pancreatic cancer sets from The Cancer Genome Atlas (TCGA) clinical and genomic information database, the top 79% of patient groups with high expression level of the FBN1 gene among pancreatic cancer patients had a worse survival prognosis than 21% of the patient group with low expression of the FBN1 gene. Of pancreatic cancer patients, the higher the expression of the FBN1, the worse the survival prognosis with a statistical significance (p<0.05) in all of overall survival (OS), disease-specific survival (DSS), progression free interval (PFI), and disease free interval (DFI).

Specifically, the survival prognosis was significantly worse in the group with high FBN1 expression (FBN1 high) than in the low group (FBN1 low). (p<0.05) In OS, p=0.02 was obtained in the comparison of 139 individuals for high FBN1 and 38 individuals for low FBN1 among 177 individuals, p=0.021 was obtained for DSS in the comparison of 136 individuals for high FBN1 and 35 individuals for low FBN1 among 171 individuals, p=0.0033 was obtained for PFI in the comparison of 142 individuals for high FBN1 and 35 individuals for low FBN1 among 177 individuals, and p=0.00018 was obtained for DFI in the comparison of 55 individuals for high FBN1 and 14 individuals for low FBN1 among 69 individuals. In the end, it was found that there was a significant difference in survival prognosis according to the level of FBN1 expression in all four survival scales.

### Example 2. Evaluation on the Level of Pancreatic Cancer Diagnostic Biomarkers in Pancreatic Cancer Cell Lines

Based on the results of Example 1, evaluation was conducted on whether asprosin is a biomarker for diagnosing pancreatic cancer. Asprosin is a protein hormone derived from profibrillin-1 produced by the FBN-1 gene, with a size of 140 amino acids formed by cleavage at the C-terminal region of profibrillin-1 by furin. To evaluate whether asprosin is a biomarker for diagnosing pancreatic cancer, the level of asprosin production was measured in five pancreatic cancer cell lines (BxPC3, MIA PaCa2, PANC1, HPAC, Aspcl) and normal pancreatic cell lines (HPNE).

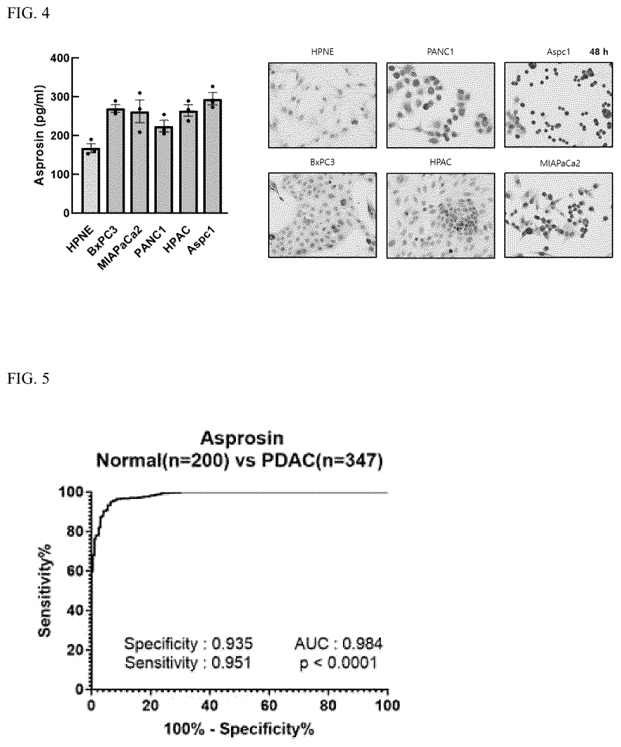

As shown in FIG. 4, five pancreatic cancer cell lines (BxPC3, MIA PaCa2, PANC1, HPAC, Aspc1) showed higher levels of asprosin than normal pancreatic cell lines (HPNE).

### Example 3. Evaluation on the Level of Pancreatic Cancer Diagnostic Biomarkers in the Blood of Pancreatic Cancer Patients

In order to evaluate whether asprosin is a biomarker for diagnosing pancreatic cancer, the level of asprosin in the blood of healthy normal individuals and pancreatic cancer patients was measured.

As shown in FIG. 5, as a result of measuring the level of asprosin in the blood of 200 normal individuals and 347 pancreatic cancer patients, it was found that the level of asprosin was statistically, significantly higher in pancreatic cancer patients than normal individuals.

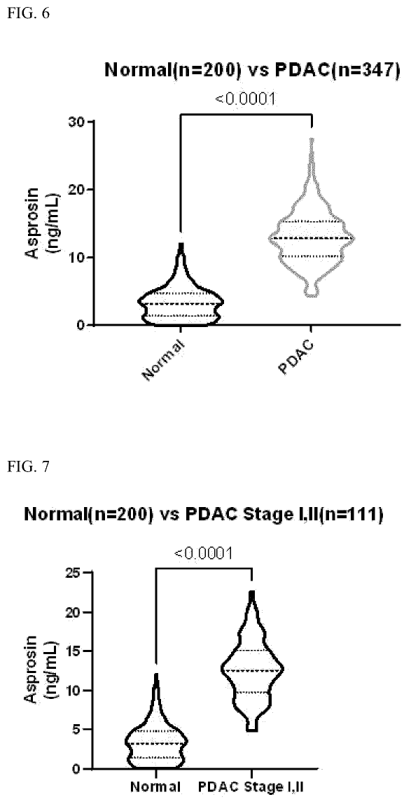

In addition, as shown in FIG. 6, as a result of analyzing the level of asprosin in the blood of 200 normal individuals and 347 pancreatic cancer patients by Receiver Operating Characteristic (ROC), the AUC value was 0.984, which is very high, in addition to very high levels of specificity of 0.935 and the sensitivity of 0.951.

### Example 4. Evaluation on the Level of Pancreatic Cancer Diagnostic Biomarkers in Early-Stage Pancreatic Cancer Patients

To evaluate whether asprosin is a biomarker for diagnosing early-stage pancreatic cancer, the level of asprosin was measured in the blood of healthy normal individuals and early-stage pancreatic cancer patients.

As shown in FIG. 7, as a result of measuring the level of asprosin in the blood of 200 normal individuals and 111 patients with early-stage pancreatic cancer (AJCC Stages 1 and 2), it was found that the level of asprosin was statistically, significantly higher in early-stage pancreatic cancer patients than the normal individuals.

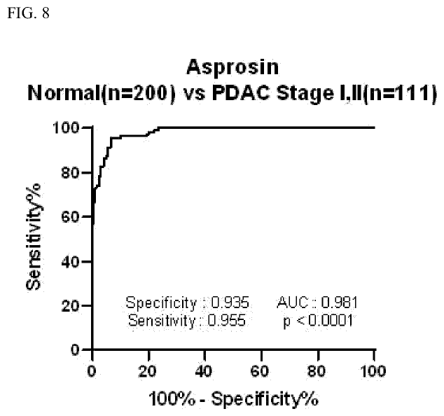

In addition, as shown in FIG. 8, as a result of analyzing the level of asprosin in the blood of 200 normal individuals and 111 patients with early-stage pancreatic cancer (AJCC Stage 1,2) by Receiver Operating Characteristic (ROC), the AUC value was 0.981, which is very high.

As described above, a specific part of the content of the present disclosure is described in detail, and for those of ordinary skill in the art, it is clear that the specific description is only a preferred embodiment and the scope of the present disclosure is not limited thereby. In other words, the substantial scope of the present disclosure may be defined by the appended claims and their equivalents.

1. A method for diagnosing pancreatic cancer in a subject, comprising:
obtaining a sample from the subject suspected of having the pancreatic cancer;
contacting the sample with a biomarker composition comprising asprosin as an active ingredient;
measuring and comparing a level of the asprosin with a control; and
diagnosing the subject having the pancreatic cancer if the level of the asprosin is higher than the control.

2. The method of claim 1, wherein the asprosin is a protein having 140 amino acids formed by cleavage at a C-terminal region in profibrillin-1.

3. The method of claim 1, wherein the asprosin has amino acids represented by SEQ ID NO: 1.

4. The method of claim 1, wherein the pancreatic cancer is an early-stage pancreatic cancer diagnosed as Stage 1 or Stage 2 by American Joint Committee on Cancer (AJCC) classification.

5. A method for detecting the expression of asprosin in a subject suspected of having pancreatic cancer, comprising:
obtaining a sample from the subject suspected of having the pancreatic cancer;
measuring a level of the asprosin by a preparation for measuring the level of the asprosin; and
comparing the expression level of the asprosin with a control.

6. The method of claim 5, wherein the preparation for measuring the level of the asprosin is an antibody that specifically binds to the asprosin.

7. The method of claim 1, wherein measuring the level of the asprosin is an antibody that specifically binds to the asprosin.

8. A method of providing information for diagnosis and prognosis assessment of pancreatic cancer, the method comprising:
measuring a level of asprosin in a biological sample derived from a subject; and
determining, if the level of the asprosin is higher than that of a healthy control group, that pancreatic cancer has developed or is likely to develop.

9. The method of claim 8, wherein the biological sample is blood, plasma, or urine.

10. The method of claim 8, further comprising: determining that survival prognosis of the subject is worse as the level of the asprosin is higher.

11. The method of claim 8, wherein the method comprises determining, if the level of asprosin during chemotherapy in the subject or after the chemotherapy appears to be lower than that before the chemotherapy, that there is improvement in the pancreatic cancer of the subject.

12. The method of claim 8, wherein the method comprises determining that a survival rate is higher as the level of asprosin is lower during chemotherapy in the subject or after the chemotherapy.


SEQUENCE LISTING (1 sequence)

SEQ ID NO: 1 - protein, 140 aa, Artificial Sequence
  Asprosin
STNETDASNIEDQSETEANVSLASWDVEKTAIFAFNISHVSNKVRILELLPALTTLTNHNRYLIESGNEDGFFKINQKEGISYLHFTKKKPVAGTYSLQISSTPLYKKKELNQLEDKYDKDYLSGELGDNLKMKIQVLLH